(19) United States
(12) Patent Application Publication
(10) Pub. No.: US 2019/0159528 A1
(43) Pub. Date: May 30, 2019

(54) A Garment

(71) Applicant: Helium 3 Resources Pty Ltd, South Perth (AU)

(72) Inventor: Raymond Dennis Palmer, South Perth (AU)

(21) Appl. No.: 16/305,871

(22) PCT Filed: Jun. 1, 2017
(86) PCT No.: PCT/AU2017/050524

§ 371 Date: Nov. 29, 2018

(30) Foreign Application Priority Data
Jun. 1, 2016 (AU) 2016902107
Jun. 22, 2016 (AU) 2016902440

(51) Int. Cl. A41C 3/00; A61N 5/06; A61N 2/02; A61N 5/04

(52) U.S. Cl. CPC A41C 3/0064; A61N 5/062; A61N 2005/067; A61N 5/04; A61N 2/02

(57) ABSTRACT

A garment, particularly in the form of a brassiere, described herein that includes a breast covering portion sized and configured to receive a breast of a wearer; and an electromagnetic radiation source facing inwardly towards the skin of the wearer, the electromagnetic radiation source configured to irradiate a portion of a breast and being positioned along a portion of the breast covering portion. Also described are methods of delivering electromagnetic radiation, and methods of treatment using the brassiere.

### FIELD

The present disclosure relates to a garment for supporting a breast of a wearer, and in particular a garment in the form of a brassiere, that has potential therapeutic application. The present disclosure also relates to methods of using the garment.

### CROSS-REFERENCE TO RELATED APPLICATIONS

This application claims the benefit of Australian Provisional Application Number 2016902107, filed 1 Jun. 2016, the subject matter of which is incorporated herein by reference in its entirety, and Australian Provisional Application Number 2016902440, filed 22 Jun. 2016, the subject matter of which is incorporated herein by reference in its entirety.

### SUMMARY

The following is a broad summary of various exemplary embodiments of a garment, and methods of use thereof. As such, it is provided solely for the purpose of enhancing the clarity of the specification. It is not intended to limit the scope of the appended claims.

In a broad form, the present disclosure is directed to a garment and more preferably a brassiere that emits electromagnetic radiation, preferably light, and more preferably light in the visible range and/or the infrared range of the electromagnetic spectrum, to a breast for therapeutic and/or cosmetic uses.

According to a first aspect of this disclosure, there is provided a brassiere. The brassiere comprises a breast covering portion sized and configured to receive a breast of a wearer; and an electromagnetic radiation source facing inwardly towards the skin of the wearer, the electromagnetic radiation source configured to irradiate a portion of a breast and being positioned along a portion of the breast covering portion.

Preferably, the electromagnetic radiation source is integrated with the breast covering portion. In other preferred embodiments, the electromagnetic radiation source is insertable into the breast covering portion. In other preferred embodiments, the electromagnetic radiation source is positionable on the outer surface of the breast covering portion.

In preferred embodiments, the electromagnetic radiation source is configured to emit a wavelength or wavelengths between about 10 nanometres and about 1 millimetre, more preferably between about 100 nanometres and about 500 micrometres, even more preferably 400 nanometres and about 100 micrometres and yet even more preferably between about 400 nanometres and about 10 micrometres. In further preferred embodiments, the electromagnetic radiation source is configured to emit a wavelength between about 10 nanometres and about 1200 nanometres, more preferably about 400 nanometres and about 1000 nanometres, even more preferably between about 630 nanometres and about 790 nanometres and yet even more preferably, a wavelength selected from the group consisting of about 630 nanometres, about 653 nanometres, about 660 nanometres and about 750 nanometres, and any combination thereof. In certain preferred embodiments, the electromagnetic radiation source is configured to emit a wavelength between about 800 nanometres to about 1200 nanometres, and more preferably, about 830 nanometres.

Preferably, the electromagnetic radiation source is selected from the group consisting of a laser, a light emitting diode, an electroluminescent panel, an arc lamp, an incandescent light source, a light emitting device and any combinations thereof. In preferred embodiments, the electromagnetic radiation source is a light emitting diode and more preferably a plurality of light emitting diode. In preferred embodiments, the light emitting diode is an organic light emitting diode or a quantum dot light emitting diode. In other preferred embodiments, the electromagnetic radiation source is a laser. In yet other preferred embodiments, the electromagnetic radiation source is an electroluminescent panel. Other preferred embodiments relate to a plurality of electromagnetic radiation sources. In certain preferred embodiments, the electromagnetic radiation source is configured to emit a radio wave. Preferably, the radio wave has a wavelength of greater than about 1 millimetre.

In preferred embodiments, the brassiere further comprises an adjustor to adjust a wavelength of electromagnetic radiation emitted from the electromagnetic radiation source. In other preferred embodiments, the brassiere further comprises a timing circuit configured to stop the electromagnetic radiation source after a predetermined amount of elapsed time. In yet other preferred embodiments, the brassiere further comprises a processor configured to adjust the frequency of the electromagnetic radiation.

In certain preferred embodiments, the brassiere further comprises a magnetic source of energy. More preferably, the level is between about 0.1 Gauss and about 100 000 Gauss.

In a second aspect, there is provided a method of delivering electromagnetic radiation to a breast tissue of a subject. The method comprises contacting a breast of the subject with a brassiere of the first aspect; and transmitting electromagnetic radiation from the brassiere to the breast tissue to thereby deliver electromagnetic radiation to the breast tissue of the subject.

In a third aspect, there is provided a method of activating a photosensitive drug in a breast tissue of a subject. The method comprises delivering the photosensitive drug to the breast tissue in need of treatment; contacting a breast with a brassiere of the first aspect; and delivering electromagnetic radiation to the breast tissue to activate the photosensitive drug, the delivering of the electromagnetic radiation including transmitting electromagnetic radiation from the brassiere to the breast tissue, to thereby activate the photosensitive drug in the breast tissue of the subject.

In a fourth aspect, the present disclosure provides a method of treating or ameliorating a breast cancer-associated symptom in a breast tissue of a subject. The method comprises contacting the breast tissue with a brassiere of the first aspect; and delivering electromagnetic radiation to the breast tissue, the delivering of the electromagnetic radiation including transmitting electromagnetic radiation from the brassiere to the breast tissue to thereby treat or ameliorate the breast cancer associated-symptom in the breast tissue of the subject.

In a fifth aspect, the present disclosure provides a method of improving a skin characteristic of a breast in a subject. The method comprises contacting at least a portion of a breast with a brassiere according to the first aspect; and delivering electromagnetic radiation to the breast, the delivering of the electromagnetic radiation including transmitting electromagnetic radiation from the brassiere to the breast to thereby improve the skin characteristic of the breast.

Suitably, the skin characteristic is selected from elasticity and fullness, and any combination thereof.

In a sixth aspect, the present disclosure provides a method of increasing a level of a connective protein in a breast tissue of a subject. The method comprises contacting the breast tissue with a brassiere according to the first aspect; and delivering electromagnetic radiation to the breast tissue, the delivering of the electromagnetic radiation including transmitting electromagnetic radiation from the brassiere to the breast tissue to thereby increase the level of the connective protein in the breast tissue.

Preferably, the connective protein is selected from elastin and collagen, and a combination thereof.

In preferred embodiments of any one of the second, third, fourth, fifth or sixth aspects, the subject is a mammal and preferably, a human.

It will be appreciated that reference herein to “preferred” or “preferably” is intended as exemplary only.

Unless the meaning is clearly to the contrary, all ranges set forth herein are deemed inclusive of the endpoints.

### BRIEF DESCRIPTION OF DRAWINGS

In order that the present disclosure may be readily understood and put into practical effect, reference will now be made to embodiments of the present disclosure with reference to the accompanying drawings, wherein like reference numbers refer to identical elements. The drawings are provided by way of example only, wherein:

FIG. 1 is a front view of a brassiere according to an embodiment of the present disclosure.

FIG. 2 is a side view of the brassiere shown in FIG. 1.

FIG. 3 is a further side of the brassiere shown in FIG. 1.

Skilled addressees will appreciate that elements in the drawings are illustrated for simplicity and clarity and have not necessarily been drawn to scale. By way of example, the relative dimensions of some of the elements in the drawings may be distorted to help improve understanding of embodiments of the present disclosure.

### DETAILED DESCRIPTION

Reference will now be made in detail to present exemplary embodiments of the present disclosure, examples of which are illustrated in the accompanying drawings.

The present disclosure is directed, at least in part, to a brassiere for treating a breast tissue of a wearer that includes a breast covering portion configured to deliver electromagnetic radiation to the breast of the wearer. The present disclosure also encompasses methods which utilise the brassiere for therapeutic and/or cosmetic purposes. It would be understood that the brassiere as presently described in not limited to a wearer of a particular gender and consequently, the brassiere applies to all genders.

The brassiere includes a breast covering portion sized and configured to receive a breast of a wearer. The breast covering portion may be part of a body of a brassiere that is configured to contact a wearer's torso. The body may extend fully or partially around the wearer's torso. Positioned along a portion of the breast covering portion is an electromagnetic radiation source facing inwardly towards the skin of the wearer, the electromagnetic radiation source being configured to irradiate a portion of a breast, as described in more detail below. Such a brassiere may be a conventional brassiere or a sport brassiere. Alternatively, the brassiere may be integrated into a garment such as a camisole. A brassiere contemplated by the current disclosure may or may not include one or more shoulder straps. The brassiere may have one or more moulded breast cups, although it is contemplated that the breasts of a wearer may be supported in a brassiere by a single unmoulded piece of fabric such as that present in some sport brassieres, for example. Preferably, the brassiere includes at least one moulded cup. It will be appreciated that the brassiere may be unitary in construction, although alternative forms of construction are contemplated.

Preferably, the breast covering portion may be a cup having a concave interior surface. More preferably, the cup is moulded so as to retain a shape when not being worn. Moulded cups may be configured to encapsulate the wearer's breasts separately. In other embodiments, a single moulded cup may encapsulate both breasts.

In certain embodiments, the brassiere may include one or more release fasteners, such as a hook and eye arrangement or a hook and loop arrangement (such as Velcro®), suitably positioned either at the front, a side or the back of the garment in order to facilitate the placement or removal of the brassiere. Contemplated within the scope of the present disclosure is a brassiere that does not include one or more release fasteners and may be positioned on a body of a wearer by placement over the head, as will be known by the skilled addressee.

The brassiere of the present disclosure may be constructed from any material that provides suitable support, comfort, elasticity, strength and/or durability, although without limitation thereto. Aesthetic appeal may be an optional consideration. The material may be natural, synthetic, or a combination thereof. Non-limiting examples of suitable natural materials include cotton and linen. Suitable synthetic materials include polyester, lycra, spandex and nylon. It is also contemplated that the brassiere may be constructed from a plurality of materials, each having a particular function or property. By way of example, a garment of the present disclosure may include a breast covering portion constructed from a highly supportive, synthetic material such as a polyester/spandex blend or lycra. The remainder of the material forming the garment may be constructed from soft, pliant material such as cotton. The brassiere may be laminated in construction.

The brassiere of the present disclosure includes an electromagnetic radiation source facing inwards towards the skin of the wearer and positioned along a portion of the breast covering portion of the brassiere. The electromagnetic radiation source is configured to irradiate at least a portion of a breast, and suitably a portion of a breast tissue of the wearer of the garment. The electromagnetic radiation source may be positioned to cover the entire surface area of a breast, or a portion of the breast, or a plurality of discrete portions of a breast. The positioning of the electromagnetic radiation source may be dependent, at least in part, on the target treatment area and the depth of target tissue, as will be appreciated by a skilled addressee. By way of example, it may be desirable to target a small and localised area on the underside of a breast and accordingly a suitably sized electromagnetic radiation source may be positioned on the lower end of a breast covering portion. Similarly, it may be desirable to position the electromagnetic radiation source on a side or the top of a breast. It is also contemplated that the electromagnetic radiation source may be positioned to target a combination of areas selected from the bottom, the top and one or both sides of a breast (and any combination thereof).

The electromagnetic radiation source may be integral, or integrated with, the breast covering portion and in particular, the inner surface of a breast covering portion. By way of example, the breast covering portion may have arrayed thereon one or a plurality of electromagnetic radiation sources. Alternatively, the electromagnetic radiation source may be insertable into a breast covering portion and in particular, insertable into a pouch or slot of a breast covering portion. In other embodiments, the electromagnetic radiation source may be attachable to a surface, and more preferably an outer surface of a breast covering portion by any suitable attachment means. The electromagnetic radiation source may be in direct contact or indirect contact with the skin of the wearer. It will be appreciated that an inner surface of a brassiere is oriented toward the breast of the wearer when worn whereas an outer surface is oriented away from the breast of the wearer when worn. It will be appreciated that the choice of wavelength emitted from the electromagnetic radiation source may be dependent, at least in part, on the depth of the targeted treatment area. Generally, although not exclusively, shorter wavelengths of less than or equal to about 700 nanometres (nm) are particularly suitable for the treatment of superficial tissue whereas wavelengths above about 700 nm penetrate further and are typically used to treat deeper-seated tissue. It will be appreciated that typically, although not exclusively, Blue light of about 400 nm to about 470 nm has depth of light penetration of <1 millimetre (mm); Blue-green light between about 475 nm to about 545 nm has depth of 0.3 mm to 0.5 mm; Yellow light of about 570 nm to about 590 nm has a depth of 0.5 mm to 2 mm; red light of about 630 nm to about 790 nm has depth of 2 to 3 mm and near infra-red of about 800 nm to about 1200 nm has a depth of 5 mm to 10 mm. The present disclosure also contemplates wavelengths in the mid infra-red and far infra-red ranges (collectively spanning a range between about 1200 nm and up to about 1 millimetre) of the electromagnetic spectrum.

In general preferred embodiments, the wavelength is preferably in the range between about 10 nm to about 1 millimetre, more preferably between about 100 nanometres and about 500 micrometres, even more preferably between about 200 nanometres and about 300 micrometres, yet even more preferably 400 nanometres and about 100 micrometres and yet even more preferably between about 400 nanometres and about 10 micrometres. In certain preferred embodiments, the electromagnetic radiation source is configured to emit a wavelength between about 400 nanometres and about 1 millimetre and more preferably, between about 800 nm and about 1 millimetre. In further general preferred embodiments, the wavelength may be in the range between about 10 nm to about 1200 nm. Preferably, the electromagnetic radiation is light in the visible range of the electromagnetic spectrum. In preferred embodiments, the wavelength of the electromagnetic radiation source is between about 400 nm and about 1000 nm, more preferably between about 400 nm and about 700 nm, even more preferably about 630 nm to about 1200 nm, even more preferably about 630 nm to about 790 nm. In other preferred embodiments, the wavelength is in a range between about 800 nm to about 1200 nm. The cited ranges include about 20 nm, about 50 nm, about 100 nm, about 200 nm, about 300 nm, about 400 nm, about 500 nm, about 600 nm, about 700 nm, about 800 nm, about 900 nm, about 1 micrometre, about 1.2 micrometres, about 10 micrometres, about 50 micrometres, about 100 micrometres, about 200 micrometres, about 300 micrometres, about 400 micrometres, about 500 micrometres, about 600 micrometres, about 700 micrometres, about 800 micrometres, about 900 micrometres and about 1 millimetre. In certain preferred embodiments, a wavelength is selected from the group consisting of about 630 nanometres, about 653 nanometres, about 660 nanometres, about 750 nanometres and about 830 nanometres, and any combination thereof. These and all ranges are to be interpreted as being fully inclusive of all values between the limits. These and all ranges are inclusive of the endpoints.

The present disclosure also contemplates a brassiere configured to emit electromagnetic radiation in the form of radio waves having a wavelength of greater than about 1 millimetre, and preferably greater than about 5 millimetres, greater than about 10 millimetres, greater than about 15 millimetres, greater than about 20, about 30, about 40, about 50, about 60, about 70, about 80, about 90, about 100 millimetres, and more.

The present disclosure contemplates brassieres and methods to irradiate a target area that uses a single wavelength (monotherapy) as well irradiating a target area with a combination of different wavelengths (so called ‘combination treatment’). Combination treatment may be useful to target various depths of tissue. Although the wavelengths are different in a combination treatment modality, other parameters such as dose, fluence, power density, pulse structure and timing may be the same or different. Such parameters will depend on the application, as will be known by a person of skill in the art. By way of example only, a combination treatment may comprise a wavelength from the red spectra and a wavelength from the near infra-red at the same or different fluence, dose, power density, pulse structure and timing. A treatment regime may include a single exposure or alternatively, a treatment course of over a number of hours, days or weeks, as is required.

The electromagnetic radiation source contemplated for use in the present disclosure is configured to emit electromagnetic radiation at one or more desired wavelengths. It will be appreciated that the present disclosure envisages inclusion of one type of electromagnetic radiation source in the brassiere or alternatively, a combination of different types of electromagnetic radiation source. Preferably, the electromagnetic radiation source is selected from the group consisting of a laser, a light emitting diode, an electroluminescent panel, an arc lamp and an incandescent light source, a light emitting device and any combinations thereof. In certain preferred embodiments, the electromagnetic radiation source uses photons at a non-thermal irradiance to alter a biological activity, otherwise termed ‘low level light therapy’. In other preferred embodiments, the electromagnetic radiation source may be a coherent light source (laser), or a non-coherent light source such as a filtered lamp or a light emitting diode (LED), or a combination of a coherent light source and a non-coherent light source. In suitable embodiments that relate to low level light therapy, the electromagnetic radiation source is a coherent light source or a non-coherent light source, or a combination thereof. According to the embodiments relating to low level light therapy, the non-coherent light source is a filtered lamp or a light emitting diode, or a combination thereof.

In preferred embodiments, the electromagnetic radiation source includes at least one light emitting diode (LED), as is known in the art. The LED device may be configured to emit one, or a plurality, of wavelengths or spectral ranges. By way of example, an LED device may be a red LED that emits only in the red spectral range. Alternatively, the LED device may be configured to emit electromagnetic radiation in the red spectral range and the near infra-red spectral range. In some preferred embodiments, the LED is an organic LED (‘OLED’). An OLED, as would be known by a skilled addressee, emits light due to electroluminescence of thin films of organic semiconductors. An OLED comprises a light emitting organic material that is suitable for use in an OLED. Preferably, the light emitting organic material is selected from a small molecule, a polymer, a dendrimer and any combinations thereof. It will be appreciated that an OLED may have a plurality of layers comprising a light emitting organic material. In an OLED, the organic semiconductors may be deposited on a glass substrate or on substrate constructed from a flexible plastic such as, but not limited to, polyethylene terephthalate. A flexible OLED offers particular advantages of being bendable and lightweight. The present disclosure also contemplates use of an inorganic LED. Preferably, the inorganic LED is a flexible LED. The garment of the present disclosure contemplates preferred embodiments a plurality of LEDs comprising at least one inorganic LED and at least one organic LED. Other embodiments relating to an LED contemplate a quantum dot LED.

Various laser or non-coherent light sources are suitable for use in the present disclosure including inert gas lasers and semiconductor laser diodes such as helium neon (HeNe; about 633 nm), ruby (about 694 nm), argon (about 488 nm and about 514 nm), krypton (about 521, about 530, about 568, about 647 nm), gallium arsenide (GaAs; >about 760 nm, with a common example of about 904 nm), and gallium aluminium arsenide (GaAlAs; about 612 to about 870 nm). Ranges are to be interpreted as being fully inclusive of all values between the limits.

Delivery of the electromagnetic radiation and in particular delivery of an electromagnetic radiation by an LED device may be either continuous or in a pulsed mode with specific pulse sequences and durations. The choice of either continuous mode or pulsed mode may be dependent on the application as is known by a skilled addressee. It is also contemplated that certain embodiments may employ a combination of a continuous mode and a pulsed mode.

Other preferred embodiments contemplate an electromagnetic radiation source in the form of an electroluminescent panel. Such panels have a benefit of being flexible and thin and may be suitable for emission of longer wavelengths. In those embodiments that contemplate an electromagnetic radiation source in the form of an arc lamp, the arc lamp is preferably a carbon arc lamp. An incandescent light source is also contemplated and may preferably be an incandescent lamp of a suitable configuration.

Any suitable power source may be included in the garment of the present disclosure. The power source may be a full battery unit integrated into the electromagnetic radiation source. Alternatively, the power source may be configured to be rechargeable, for example by inclusion of an AC power socket or a USB socket. Also contemplated are renewable energy sources such as solar power. It will be appreciated that the power source may be configured to suit a particular electromagnetic radiation source, as will be known by a person of skill in the art.

Preferred embodiments relating to the garment of the present disclosure include a timing circuit configured to stop the electromagnetic radiation after a predetermined amount of elapsed time. Such a timing circuit may be particularly advantageous for a pulsed mode of operation. For example, a predetermined amount of elapsed period of time may be about 10 seconds, about 20 seconds, about 30 seconds, about 40 seconds, about 50 seconds, and about 60 seconds or more, 1 hour or 2 hours. The predetermined amount of elapsed time may be dependent on the application.

The present disclosure contemplates embodiments that may include an adjustor to adjust a wavelength of electromagnetic radiation emitted from the electromagnetic radiation source. This is advantageous if a plurality of wavelengths is desired. By way of example only, the garment may include a red LED device configured to emit light at any wavelength in the red spectral range and an adjustor for adjusting to a wavelength between about 630 nm and about 700 nm accordingly. A conventional adjustor may be contemplated and may include a processor.

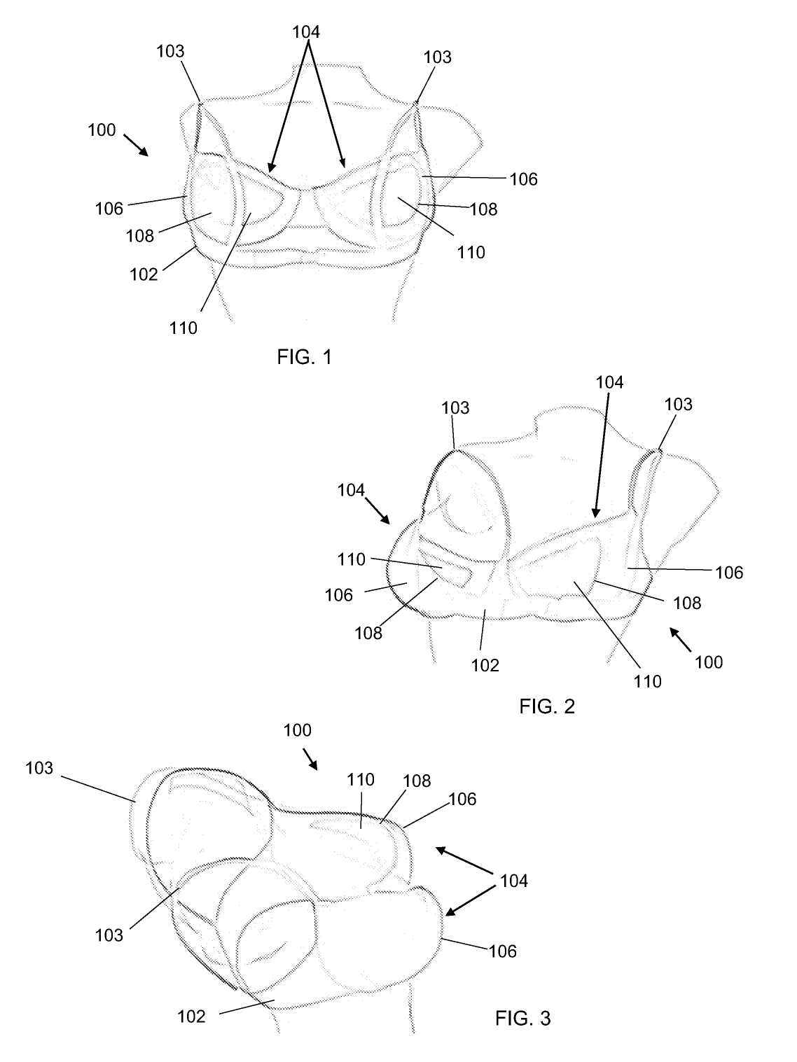

Reference is made to FIGS. 1 to 3, wherein reference numeral 100 generally indicates a brassiere according to an exemplary embodiment of the present disclosure. Brassiere 100 includes a body 102 extending fully around the torso of a wearer, and straps 103 configured to lie over a shoulder of the wearer. Body 102 includes two cups 104, and preferably moulded cup portions, having a concave interior surface. Cup 104 includes a breast covering portion 106 sized and configured to receive a breast of the wearer. Positioned along a portion 108 of the breast covering portion 106 is an electromagnetic radiation source 110 facing inwardly towards the skin of the wearer. Electromagnetic radiation source 110 is configured to irradiate a portion of the wearer's breast as herein described. According to this embodiment, electromagnetic radiation source 110 is engaged with breast covering portion 106 to thereby cover a portion of the wearer's breast.

The present disclosure contemplates methods, including therapeutic and/or cosmetic methods, that deliver or transmit electromagnetic radiation from the brassiere as described herein.

The methods of the disclosure discussed herein include methods that includes exposing or delivery of electromagnetic radiation to a breast tissue of a subject. The term “breast tissue” as used herein is inclusive of all tissues relating to a breast or a breast component including cutaneous (i.e. dermal) tissue, subcutaneous tissue, muscular tissue, adipose tissue, lymph node and vessels, mammary ducts, fibrous tissue, mammary glands, areola, connective tissue, blood vessels, stromal tissue, cysts and glandular tissue. In certain embodiments, the breast tissue or breast component may include a neoplasm or other type of cancerous growth. Therefore, in other preferred embodiments, the breast tissue may be a cancerous breast tissue. The cancerous breast tissue may be malignant or benign. It will be appreciated that the garments and methods of the disclosure including irradiating or treating a plurality of breast tissue.

The terms “patient”, “subject”, “host” or “individual” used interchangeably herein, refer to any subject, particularly a vertebrate subject, preferably an animal subject and even more particularly a mammalian subject, for whom therapy or prophylaxis is desired. Preferably, the mammalian subject is a human.

In particular aspects, methods of the present disclosure include activating a photosensitive drug in a breast tissue which includes the steps of delivering the photosensitive drug to the breast tissue in need of treatment; contacting a breast with a brassiere as described herein and delivering electromagnetic radiation to the breast tissue to activate the photosensitive drug, the delivering of the electromagnetic radiation includes transmitting electromagnetic from the brassiere to the breast tissue as herein described. Preferably, methods of activating a photosensitive drug use visible red light emitted a laser or other suitable light source such as LED. Suitable examples of a photosensitive drug include porfimer sodium, aminolevulinic acid, a methyl ester of aminolevulinic acid and 2-[1-hexyloxyethyl]-2-devinyl pyropheophorbide-a (Photochlor). It will be appreciated that such methods include photodynamic therapy (PDT), being a treatment for cancer that utilises light to activate photosensitising agents that have been absorbed by the cancer cells. The present disclosure is particularly suited for use in photodynamic therapy for the treatment of cancerous growths, solid tumours and metastatic cancers, although without limitation thereto. Agostinis et al. (2011, CA Cancer J Clin 61(4):250-281) provides an informative review of photodynamic therapy of cancer and is incorporated herein by reference.

Methods of the disclosure include methods of treating or ameliorating a breast cancer-associated symptom. As used herein, the terms “treatment,” “treating,” and the like, refer to obtaining a desired pharmacologic and/or physiologic effect. The effect may be therapeutic in terms of a partial, or complete, remediation of symptom. These terms also cover any treatment of a symptom in a subject and include: (a) inhibiting of the symptom, i.e., arresting its development; or (b) relieving the symptom, i.e., causing regression of symptom.

A “breast cancer-associated symptom” as used herein includes a lump in the form of a growth or a cyst in a breast tissue, a skin change of a breast such as, but not limited to, redness or dimpling, a change in the appearance of an areola such as crusting or ulceration, a change in a breast shape and/or size and appearance of a lump in an armpit. It is contemplated that a breast cancer-associated symptom may include one or more genomic or genetic mutations associated with development of a breast cancer. Such mutations may or may not manifest as an anatomical symptom such as a cancerous growth.

Methods of treating or ameliorating a breast cancer-associated symptom as disclosed herein may further include therapeutic treatment with a drug or treatment regime such as hormone therapy, antibody therapy, chemotherapy and the like.

The present discloses contemplates cosmetic methods to improve appearance of at least one skin characteristic of a breast by exposing a breast or delivering to the breast, electromagnetic radiation using a brassiere as described herein. These methods also contemplate improving a skin characteristic of a breast by delivering electromagnetic radiation to a breast tissue to improve a skin characteristic of a breast. A skin characteristic may be an improvement in elasticity which has aesthetically appears a skin tightness and/or firmness. Another skin characteristic may be fullness or plumpness. By “improve” or “improvement”, it is meant an increase, promotion, benefit of a skin characteristic when exposed to electromagnetic radiation emitted from the brassiere according to the present disclosure when compared to a breast not exposed to electromagnetic radiation emitted from the brassiere. The improvement may be a 5%, 10%, 20%, 30%, 40%, 50%, 60%, 70%, 80%, 90% or up to 100% increase in a skin characteristic when compared to a breast not exposed to electromagnetic radiation emitted from the brassiere of the present disclosure.

The present disclosure contemplates increasing a level of a connective protein in a breast tissue which includes using the brassieres as described herein. Increasing a level may be a protein level, a level of protein synthesis, a transcript level such as mRNA, a level of an active splice variant, a level of a functional activity of a connective protein, an increase in a level of a precursor protein (e.g., procollagen or tropoelastin) and a protein turnover or protein stability level, and any combination thereof. Suitably, the connective protein is elastin or collagen, or a combination thereof. According to preferred embodiments of this aspect, the wavelength is between about 630 nm to about 1200 nm, more preferably 630 nm to about 700 nm. In those embodiments that contemplate increasing a level of a collagen, preferably type I collagen, the preferred wavelength is selected from 630 nm, about 660 nm and about 830 nm, and any combination thereof. Ranges are to be interpreted as being fully inclusive of all values between the limits and are inclusive of the endpoints.

The present disclosure contemplates embodiments which further comprise sources or dosages of energy other than those embodied by the electromagnetic spectrum. In some preferred embodiments, energy derived from a magnetic source may be included. It is contemplated that in preferred embodiments, a magnetism level of up to and including about 100 000 Gauss (and all integers in-between) may be used and preferably, between about 0.1 Gauss and about 100 000 Gauss (and all integers in-between). In other preferred embodiments, the magnetism level may be between about 100 Gauss and about 50 000 Gauss, more preferably between about 5000 Gauss and about 25 000 Gauss. These ranges are to be interpreted as being fully inclusive of all values between the limits. These ranges are inclusive of the endpoints. In certain preferred but non-limiting embodiments, a magnetism level of 800 Gauss (0.08 Tesla) for 25 minutes is preferred.

As will be appreciated by a skilled addressee, if the light applied is not of sufficient irradiance or the irradiation time is too short then there is no response. If the irradiance is too high or irradiation time is too long, then the response may be inhibited. Somewhere in between may be the optimal combination of irradiance and time for stimulation. It will be appreciated that parameters such as duration of exposure (can be known as irradiation time), power of electromagnetic radiation (mW), energy (J) of electromagnetic radiation, suitable type of electromagnetic radiation source, number of treatments or exposures, treatment interval (hours, days or weeks), energy density (J/cm²), wavelength of electromagnetic radiation and depth of penetration of electromagnetic radiation is dependent, at least in part, upon the method being employed and as will be known by a person of skill in the art. Typically, although not exclusively, in those embodiments that contemplate low level light therapy, relatively low fluences (between about 0.04 to about 50 J/cm²) and power densities (<100 mW/cm²) may be used. Ranges are to be interpreted as being fully inclusive of all values between the limits.

The following prophetic example is made: A treatment using the brassiere of the present disclosure will be irradiance (Intensity) at 1 milli watts per cm², the wavelengths that will be used are wavelength 1 of 653 nm and Wavelength 2 of 750 nm for treatment duration of 2 hours. This prophetic example may further include a magnetic dose of 800 Gauss (0.08 Tesla) for 25 minutes.

The appended claims are to be considered as incorporated into the above description.

Throughout the specification, including the claims, where the context permits, the term “comprising” and variants thereof such as “comprise” or “comprises” are to be interpreted as including the stated integer or integers without necessarily excluding any other integers.

It is to be understood that the terminology employed above is for the purpose of description and should not be regarded as limiting. The described embodiments are intended to be illustrative of the present disclosure, without limiting the scope thereof. The present disclosure is capable of being practised with various modifications and additions as will readily occur to those skilled in the art.

Various substantially and specifically practical and useful exemplary embodiments of the claimed subject matter, are described herein, textually and/or graphically, including the best mode, if any, known to the inventors for carrying out the claimed subject matter. Variations (e.g., modifications and/or enhancements) of one or more embodiments described herein might become apparent to those of ordinary skill in the art upon reading this application. The inventors expect skilled artisans to employ such variations as appropriate, and the inventors intend for the claimed subject matter to be practiced other than as specifically described herein. Accordingly, as permitted by law, the claimed subject matter includes and covers all equivalents of the claimed subject matter and all improvements to the claimed subject matter. Moreover, every combination of the above described elements, activities, and all possible variations thereof are encompassed by the claimed subject matter unless otherwise clearly indicated herein, clearly and specifically disclaimed, or otherwise clearly contradicted by context.

The use of any and all examples, or exemplary language (e.g., “such as”) provided herein, is intended merely to better illuminate one or more embodiments and does not pose a limitation on the scope of any claimed subject matter unless otherwise stated. No language in the specification should be construed as indicating any non-claimed subject matter as essential to the practice of the claimed subject matter.

Thus, regardless of the content of any portion (e.g., title, field, background, summary, description, abstract, drawing figure, etc.) of this application, unless clearly specified to the contrary, such as via explicit definition, assertion, or argument, or clearly contradicted by context, with respect to any claim, whether of this application and/or any claim of any application claiming priority hereto, and whether originally presented or otherwise:
a. there is no requirement for the inclusion of any particular described or illustrated characteristic, function, activity, or element, any particular sequence of activities, or any particular interrelationship of elements;
b. no characteristic, function, activity, or element is “essential”;
c. any elements can be integrated, segregated, and/or duplicated;
d. any activity can be repeated, any activity can be performed by multiple entities, and/or any activity can be performed in multiple jurisdictions; and
e. any activity or element can be specifically excluded, the sequence of activities can vary, and/or the interrelationship of elements can vary.

The use of the terms “a”, “an”, “said”, “the”, and/or similar referents in the context of describing various embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Moreover, when any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate subrange defined by such separate values is incorporated into the specification as if it were individually recited herein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

Words indicating direction or orientation, such as “front”, “rear”, “back”, etc., are used for convenience. The inventor(s) envisages that various embodiments can be used in a non-operative configuration, such as when presented for sale. Thus, such words are to be regarded as illustrative in nature, and not as restrictive.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

1. A brassiere, the brassiere comprising:
a breast covering portion sized and configured to receive a breast of a wearer; and
an electromagnetic radiation source facing inwardly towards the skin of the wearer, the electromagnetic radiation source configured to irradiate a portion of a breast and being positioned along a portion of the breast covering portion.

2. The brassiere of claim 1, wherein the electromagnetic radiation source is integrated with the breast covering portion.

3. The brassiere of claim 1, wherein the electromagnetic radiation source is insertable into the breast covering portion.

4. The brassiere of claim 1, wherein the electromagnetic radiation source is configured to emit a wavelength between about 10 nanometres and about 1 millimetre.

5-13. (canceled)

14. The brassiere of claim 1, wherein the electromagnetic radiation source is selected from the group consisting of a laser, a light emitting diode, an electroluminescent panel, an arc lamp, an incandescent light source, and any combinations thereof.

15. The brassiere of claim 14, wherein the electromagnetic radiation source is a light emitting diode.

16. The brassiere of claim 15, wherein the light emitting diode is an organic light emitting diode.

17. The brassiere of claim 16, wherein a light emitting organic material of the organic light emitting diode is selected from the group consisting of a small molecule, a polymer, a dendrimer, and any combinations thereof.

18. The brassiere of claim 15, wherein the light emitting diode is a quantum dot light emitting diode.

19. The brassiere of claim 14, wherein the electromagnetic radiation source is an electroluminescent panel.

20. The brassiere of claim 14, wherein the electromagnetic radiation source is a laser.

21. The brassiere of claim 14, wherein the electromagnetic radiation source is an arc lamp.

22. The brassiere of claim 14, wherein the electromagnetic radiation source is an incandescent light source.

23. The brassiere of claim 1, further comprising an adjustor to adjust a wavelength of electromagnetic radiation emitted from the electromagnetic radiation source.

24. The brassiere of claim 1, further comprising a timing circuit configured to stop the electromagnetic radiation source after a predetermined amount of elapsed time.

25. The brassiere of claim 1, further comprising a processor configured to adjust the frequency of the electromagnetic radiation.

26. The brassiere of claim 1, further comprising a magnetic source of energy.

27. The brassiere of claim 26, wherein the magnetic source of energy is a level between about 0.1 Gauss and about 100 000 Gauss.

28-36. (canceled)
